(19) United States
(12) Patent Application Publication
(10) Pub. No.: US 2015/0105319 A1
(43) Pub. Date: Apr. 16, 2015

(54) DURABLE HAEMOSTATIC SCAFFOLD

(71) Applicants: Paul JANMEY, Media, PA (US); Raivo UIBO, Tartu (EE); Peep VESKI, Tartu (EE); Ivo LAIDMÄE, Tartumaa (EE)

(72) Inventors: Paul JANMEY, Media, PA (US); Raivo UIBO, Tartu (EE); Peep VESKI, Tartu (EE); Ivo LAIDMÄE, Tartumaa (EE)

(21) Appl. No.: 14/577,083

(22) Filed: Dec. 19, 2014

Related U.S. Application Data
(62) Division of application No. 13/977,127, filed on Sep. 6, 2013.

(30) Foreign Application Priority Data
Dec. 29, 2010 (EE) P20100093

(51) Int. Cl. A61L 27/22; A61L 27/56; A61L 27/58; A61L 27/20

(52) U.S. Cl. CPC A61L 27/225; A61L 27/20; A61L 27/56; A61L 27/58; A61L 2430/00; A61L 2400/04; A61L 2300/404; A61L 2300/418
USPC 514/13.6

(57) ABSTRACT

A method for preparing a haemostatic and adhesive durable scaffold useful for promoting wound healing, and the scaffold so prepared, are provided. The scaffold made of fibrinogen and chitosan is produced by electrospinning techniques, resulting in a material with enhanced endurance, applicable in various medical purposes including surgery, tissue regeneration, burns, injuries, and the like. The scaffold is produced in the absence of biocatalysts, in particular thrombin.

### FIELD OF THE INVENTION

This invention relates to medicine, in particular to materials useful for tissue regeneration and haemostatic compositions in the prevention of blood loss from injuries, surgical procedures and traumatic wounds.

### BACKGROUND OF THE INVENTION

Tissue engineering and wound healing have been quickly evolving interdisciplinary areas which have been of great interest for decades. Ideally wound healing products should be safe (low immunogenicity, free from infectious agents like prions and viruses), effective and low cost. During the 1980's, increased awareness of the HIV and hepatitis, use of unpurified blood and blood products hampered the development of safe and effective fibrinogen-based haemostatic dressings. Developments on recombinant protein technology and improvements in plasma purification methods started reversing that trend. However, the structural complexity of fibrinogen makes the production of recombinant proteins impractical or costly. Current sources of fibrinogen used in fibrin based wound healing products are limited to pooled mammalian blood products. Contamination with bacteria, viruses or prions accompanied by the risk of infecting the patient has been considered to be a barrier constraining the widespread approval of many potential applications.

A need in the materials exists, which could be used to form biocompatible dressings that possess structural integrity, good haemostatic properties and subsequently support angiogenesis and tissue reparation and also possesses good adhesion properties. Both synthetic and natural polymers are used in tissue engineering and wound healing. Natural polymers like collagen, fibrinogen, chitosan, or structurally similar biocompatible polymers have been of great interest during last decades.

Hundreds of synthetic and natural polymers have been processed into nanofibres by electrospinning, including fibrinogen, collagen, and chitosan. Although general principles of electrospinning in the preparation of the nanofibre mats are known, every polymer needs well-specified conditions to get a most appropriate product for medical use. A number of methods exist for manufacturing extracellular matrix (ECM) like scaffolds. Electrospinning is a scaffold manufacturing technique that produces interconnected nanofibres in a continuous manner. Fibre diameter can range from 3 nm to several micrometers depending on numbers of parameters. Scaffolds made by electrospinning mimic closely natural ECM by possessing properties like high surface area, high porosity and small pore size.

Fibrin-based biomaterials are biocompatible and biodegradable and have high affinity to various biological surfaces. Being a naturally occurring physiological scaffold, it supports angiogenesis and tissue repair. In addition, fibrin naturally contains sites for cellular binding, and has been shown to have excellent cell seeding effects and good tissue development.

Pure fibrinogen based dressing or scaffold is fragile and with poor mechanical properties. Therefore fibrinogen or other blood clotting species in a dressing are typically in multilayered setting and often used as lyophilized onto the material that serves as the haemostatic dressing backing-support layer.

Fibrinogen has been processed into fibres by electrospinning from 1,1,1,3,3,3-hexafluoroisopropanol solutions. Besides being soluble in water, proteins are often soluble in perfluorinated alcohols such as 1,1,1,3,3,3-hexafluoroisopropanol, and 2,2,2-trifluoropropanol. The acute toxicity of 1,1,1,3,3,3-hexafluoroisopropanol, however, is well documented. For example U.S. Pat. No. 7,615,373 for electrospun collagen and U.S. Pat. No. 7,759,082 for electroprocessed fibrin.

Chitosan is a positively charged polysaccharide composed of β(1-4)-linked d-glucosamine monosaccharides with randomly interspersed N-acetylglucosamine. Chitosan is a natural compound, non-toxic to tissues. It is a biodegradable and bioadhesive polymer with bacteriostatic, fungicidal characteristics. A number of studies present chitosan in wound treatment applications, in tissue engineering applications as cartilage tissue, bone substitutes, respiratory epithelial cells for a possible tissue engineered trachea or in nerve cell attachment and proliferation experiments. It has been reported that chitosan acts as chemo-attractant to macrophages and neutrophiles in wound healing process. Chitosan accelerates the tensile strength of wounds by speeding the fibroblastic synthesis of collagen in the initial phase of wound healing. Chitosan has an analogous structure with glycosaminoglycan, which is one of the main components of natural ECM. However, based on an in vitro study protocol, chitosan scaffolds did not support human dermal fibroblast (HDF) attachment (K. W. Ng, H. L. Khor, D. W. Hutmacher. In vitro characterization of natural and synthetic dermal matrices and culture with human dermal fibroblasts. Biomaterials 25 (2004) 2807-2818).

Haemostatic agents comprising fibrinogen and chitosan have been described in prior art. U.S. Pat. No. 5,773,033 (Cochrum et al. Fibrinogen/chitosan hemostatic agents) describes a haemostatic material composed of chitosan and fibrinogen, wherein fibrinogen is obtained by ammonium sulphate precipitation. The fibrin of the agent is obtained by the catalytic activity of thrombin and other platelet-derived factors.

WO2007135492 (Larsen et al, Methods for making a multicomponent hemostatic dressing) provides a multicomponent dressing for wound healing, but it comprises an additional step of coating the obtained scaffold of a polymeric substance with biologically active protein component(s), which covers the original scaffold.

WO2006019600 (Shalaby et al, Hemostatix microfibrous constructs) provides a method for producing haemostatic fibrous construct of polymeric substances by electrospinning, but it relates mainly to constructs exhibiting “core and sheet” character.

The capability to electrospin a polymer is dependent upon finding the optimal solvent system, among optimizing many other parameters.

No dressings or matrices consisting entirely of separate chitosan and fibrinogen nonwoven nano- or microfibers (uniformly distributed along matrix) made by one step electrospinning of mentioned polymers mixture (suspension) have been described. However, eliminating the need for biological catalyzers, moreover for mammal-derived factors, would facilitate the production of the agents and increase their biological safety. In case of multicomponent agents, where the separately manufactured fibrous bioabsorbable scaffold is coated with required blood-clotting or coagulation inducing proteins, the favourable properties of the bioabsorbable scaffold will be masked.

Use of external thrombin, either human recombinant or heterologous (most commonly of bovine origin) for supporting the polymerization of fibrinogen is problematic due to the induction of antibodies against thrombin impairing so the normal coagulation process. Furthermore, the administration of external thrombin may be directly lifethreatening in cases of pre-existing autoantibodies against thrombin, demonstrated in different rheumatic and autoimmune disorders, including antiphospholipid syndrome with high propensity for the stroke and myocardial infarction. In a study of Ballard et al. (J Am Coll Surg 2010; 210:199-204) as much as 2.2% of surgery patients had antibodies to human recombinant thrombin without previous expose to this preparation. External thrombin may also induce anaphylactic reactions (Tadokoro et al., J Allergy Clin Immunol 1991; 88:620-629; Wuthrich et al., Allergy 1996; 51:49-51).

Thus, there exists a need in the art for material that possesses haemostatic characteristics of fibrinogen, adhesion properties of chitosan, good mechanical properties (enhanced endurance) and which can be obtained without using biochemical catalysts, in particular thrombin.

The mentioned shortcomings are avoided in the current invention.

### BRIEF DESCRIPTION OF DRAWINGS

FIG. 1. Scanning Electron Microscopy micrographs of electrospun chitosan (3.5%) and fibrinogen (125 mg/ml) from blended solution. Magnification: A-3000×, picture width corresponds to 27.4 μm; B-5000×, picture width corresponds to 16.4 μm.

FIG. 2. Scanning Electron Microscopy micrographs of electrospun salmon fibrinogen dissolved in HFP/TFA solution at the concentration of 60 mg/ml (A,B) and 125 mg/ml (C,D). Magnification: A,C-3000×, picture width corresponds to 27.4 μm; B,D-5000×, picture width corresponds to 16.4 μm.

FIG. 3. Scanning Electron Microscopy micrographs of electrospun 6% chitosan in TFA.

Magnification: A-10000×, picture width corresponds to 8.2 μm; B-20000×, picture width corresponds to 4.1 μm.

Feeding rate 1 ml/h and voltage 1 KV/cm were best conditions for 6% chitosan/TFA solution electrospinning.

FIG. 4. Wound areas at the last day of the experiment after treatment with different types of dressings photographed by digital camera. Rat no. 15; Day 10.

A—Control, B—Fibrinogen+Chitosan, C—Fibrinogen, D—Chitosan

FIG. 5. Wound healing percentage at the end of experiment (day 10).

FIG. 6. In vitro proliferation (metabolic activity by MTS assay) of fibroblasts cultured on the scaffold for 2-14 days.

FIG. 7. Fluorescence micrograph of fibroblasts on fibrinogen-chitosan scaffold after 14 days. Construct is fixed with formalin and stained with DAPI and phalloidin conjugated with FITC.

### DESCRIPTION OF THE INVENTION

The object of the present invention is a durable haemostatic scaffold with enhanced bioadhesivity comprising simultaneously electrospun fibrinogen and/or fibrin, and chitosan.

The fibres of electrospun chitosan and fibrinogen/fibrin both are exposed to the inner and outer surface of the scaffold, and the gaps in the scaffold are of size sufficient for eukaryotic cells to attach and proliferate, in result forming a haemostatic and adhesive structure with enhanced durability and bioadhesivity.

Said scaffold is produced by simultaneous electrospinning of a mixture of fibrinogen/fibrin and chitosan solutions in the absence of biocatalysts enabling the polymerization reaction of fibrinogen into fibrin, in particular thrombin. The new scaffold provided in the present invention can be used for promoting tissue growth. The present invention avoids the use of layers of different substances and offers the potential to incorporate the polymer compounds directly in the dressing. Any other components promoting wound healing or possessing favourable effects on tissue regeneration can be added to the scaffold. Simultaneous electrospinning enables the characteristics of both (or more) components to be exposed to the outer surface of the obtained scaffold, hereby providing the good adhesion to the wounds of chitosan as well as cellular binding, cell seeding effects, support of angiogenesis of fibrin (fibrinogen) to be involved in the processes of wound healing and tissue regeneration. The scaffold made by electrospinning can be used as a haemostatic and wound healing bandage, tissue sealant for different internal injuries (incl. brain/spinal cord injuries), substrate for supporting allogenic cell growth in animal or human tissues, substrate in introducing plasmids with DNA for gene therapy and substrate for other biotherapies (introduction of immune cells, dendritic cells, stem cells etc.).

Moreover, the method known in the prior art providing a scaffold of chitosan (exhibiting low bioadhesivity), which is coated by dipping it into a solution of fibrinogen, does not eliminate the need of catalysing the polymerization reaction of fibrinogen to fibrin. This kind of coating obviously reduces the space of the gaps between the fibres, thus depleting the space required for eukaryotic cells to migrate into the scaffold to form a basement for regeneration of the injured tissue.

On the contrary, in the scaffold provided in the invention, sufficient gaps are maintained in the scaffold to enable eukaryotic cells to migrate into the scaffold to form a basement for regeneration of the injured tissue. This kind of scaffold is useful also in tissue and organ culture, and may prove as a structural framework for tissue and organ modelling.

As an additional feature of the invention, the scaffold is obtained in a one-step process, eliminating the stage of coating.

As a major advantage of the invention, the need for biocatalyzation of the process of polymerization of fibrinogen into fibrin is eliminated, thus providing high safety for medical and veterinary use, as the risk for biological contamination from biocatalyzers like thrombin, is reduced.

This kind of material is a promising means for various applications in human medicine and veterinary medicine, including but not limited to, wound healing, surgery, tissue regeneration, supporting drug delivery, gene therapy, tissue and organ (re)modelling, treatment of injuries and burns. The scaffold may additionally contain antimicrobials, antiseptics, anesthetics, analgesics, wound healing agents, anti-inflammatory compounds, antiviral agents and growth promoters. This material can be also applied to cell, tissue and organ cultures as a supporting scaffold for cell attachment.

The fibrinogen used in manufacturing the scaffold is obtained from non-mammalian vertebrates. In a more preferred embodiment the fibrinogen used in manufacturing the compound scaffold is obtained from fish, in particular from salmon.

The compounds of the scaffold are to be solved in appropriate solvents. In an embodiment of the invention, the scaffold is obtained by simultaneous electrospinning of fibrinogen and chitosan solutions, wherein the solvents of fibrinogen and chitosan are halogenated alcohols and acids. In particular, fibrinogen is dissolved in a mixture of halogenated alcohol(s) and halogenated acid(s), chitosan is dissolved in halogenated acid(s).

In a preferred embodiment of the invention, the scaffold is obtained by simultaneous electrospinning of fibrinogen solution in a mixture of hexafluoropropanol/trifluoroacetic acid and the solution of chitosan in trifluoroacetic acid. In a more preferred embodiment of the invention, the solvent of fibrinogen solution is hexafluoropropanol/trifluoroacetic acid (90:10) and the solvent of chitosan is 100% trifluoroacetic acid.

Simultaneous electrospinning of the solutions of fibrinogen and chitosan is carried out at the voltage biases from 5 to kV, preferably at 10 kV. The haemostatic and adhesive scaffold with enchanced endurance comprising simultaneously electrospun fibrinogen and/or fibrin, and chitosan, accompanies characteristics of fibrinogen by acting as haemostatic and subsequently supports angiogenesis and tissue repair and having better adhesion properties than chitosan alone. Chitosan in combination with fibrinogen in scaffold improves mechanical properties (enhanced endurance) that are poor in electrospun fibrinogen alone.

### DESCRIPTION OF THE EMBODIMENTS

### Example 1

### Electrospinning of Fibrinogen and Chitosan

Lyophilised salmon fibrinogen was dissolved in the solution of hexafluoropropanol (HFP) and trifluoroacetic acid (TFA) (90:10). The final concentration of fibrinogen was 125 mg in 1 ml HFP/TFA solution.

Chitosan (with molecular weight of ca 130 KDa) was dissolved in pure TFA overnight. The final concentration of chitosan was 0.035 g in 1 ml of TFA.

0.5 ml of fibrinogen solution and 0.5 ml of chitosan solution were sucked into a 1 ml syringe and blended. A capillary was attached to the syringe and electrospinning was performed.

10 KV voltage was applied to the capillary, the solution was ejected from the syringe at the speed of 0.5 ml/h, and the grounded target was kept at the distance of 7 cm from the syringe.

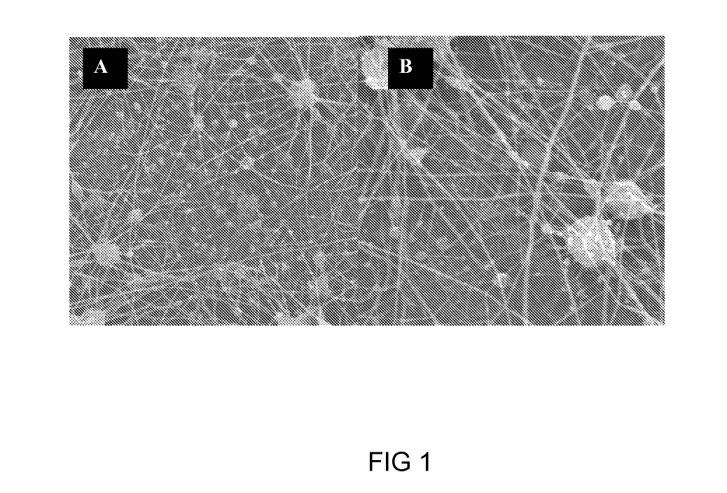

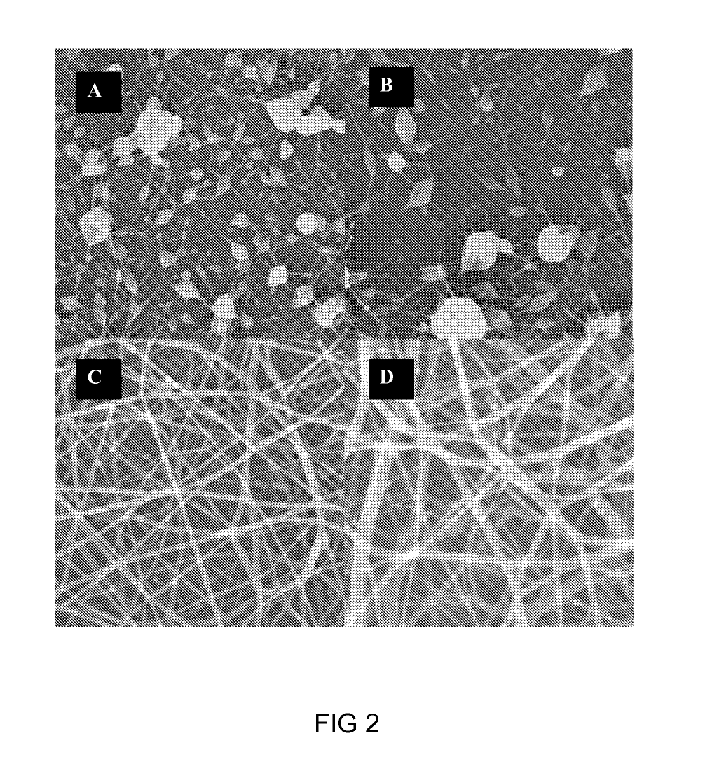

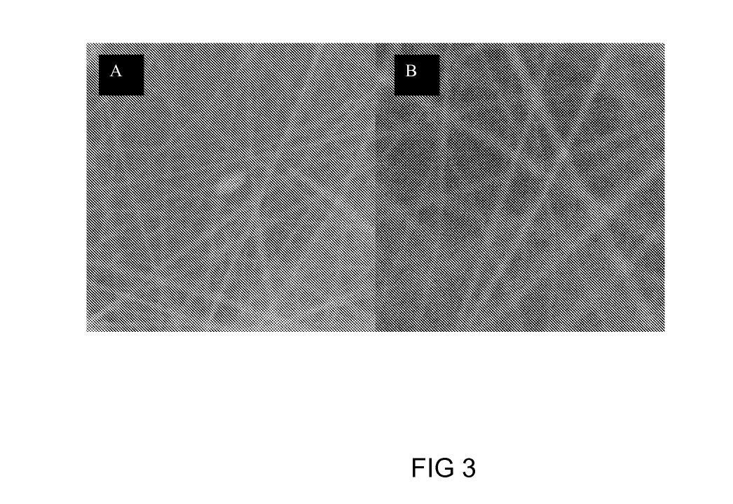

The obtained scaffold was analysed by Scanning Electron Microscopy (SEM)(FIG. 1) and subjected to further experiments. In parallel, (SEM) was performed to scaffolds obtained by electrospun fibrinogen (FIG. 2) and chitosan solutions (FIG. 3).

In FIG. 1 it is seen, that visually observable features from both chitosan and fibrinogen scaffolds are present in the scaffold obtained by electrospinning of the mixture of the solutions of fibrinogen and chitosan.

### Example 2

### Electrospun Chitosan-Fibrinogen Scaffold for Wound Repair

The scaffold of the invention of salmon fibrinogen and chitosan was prepared by electrospinning as previously described. For the control scaffolds, salmon fibrinogen only and chitosan only were spun at the same conditions as for the preparation of the scaffold of the current invention.

Materials containing chitosan were further neutralized by immersing the membranes in 5M NaOH aqueous solution for 1 hour. After the immersion the scaffolds were washed with distilled water until neutral pH was reached.

Ten 21 weeks old Wistar rats (10 males) were used. The animals were obtained from Harlan CPB (The Netherlands) and raised in the Vivarium of the Biomedicum at the University of Tartu (Tartu, Estonia). All animals were kept in the same standard conditions in the same room. Approval for the animal experimentation was obtained from the committee of animal experimentation (Estonian Ministry of Agriculture). Anesthesia was induced in rats by injection of ketamine (83 mg/kg of Bioketan, lot 9A1514D, Vetoquinol Biowet, Gorzow Wlkp., Poland) and xylasin (6.67 mg/kg of Xylapan, lot 010709D, Vetoquinol Biowet, Gorzow Wlkp., Poland) intraperitoneally. Skin of the back of every animal was prepared for septic dissection (hairs were shaved and skin surface was treated with 70% ethanol). After the drying of the skin, split-thickness skin grafts were removed from four sides (approx. area—1.5 cm×1.5 cm each) of the rat back using hand dermatome from E. Weck & Co. Blades (pilling weckprep, cat. No. 450205) used in dermatome are from TFX Medical Ltd., UK.

All prepared wound dressing scaffolds were stored at room temperature not more than 3 days before application to the wound area. Prior to application to the wound, the scaffolds were neutralized and sterilized by soaking in 70% ethanol (1 hour). Thereafter the scaffolds were repeatedly washed with sterile PBS. Excess of the PBS was removed by patting the scaffolds with dry gauze and after that the scaffold was placed on the wound and was hold in place with dry gauze for 1 minute. No further wound dressing was used.

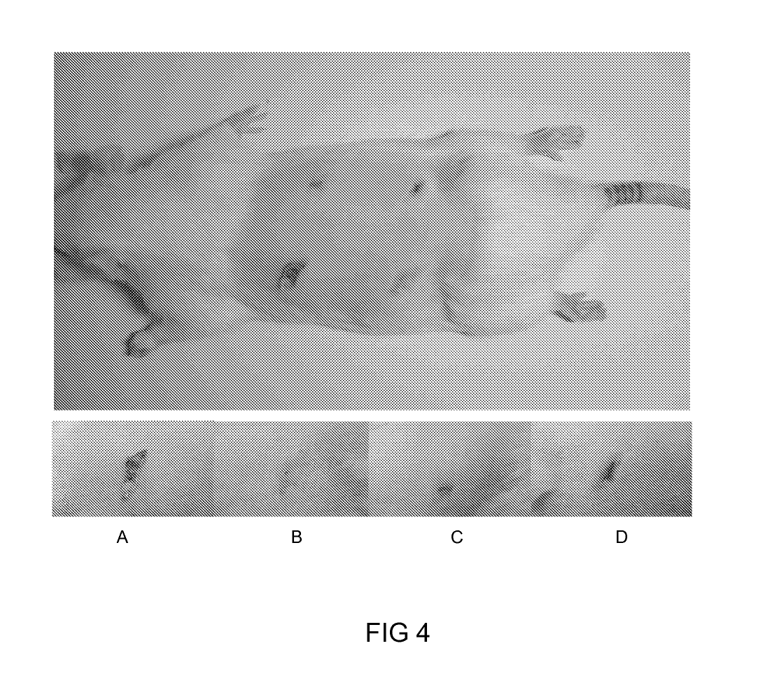

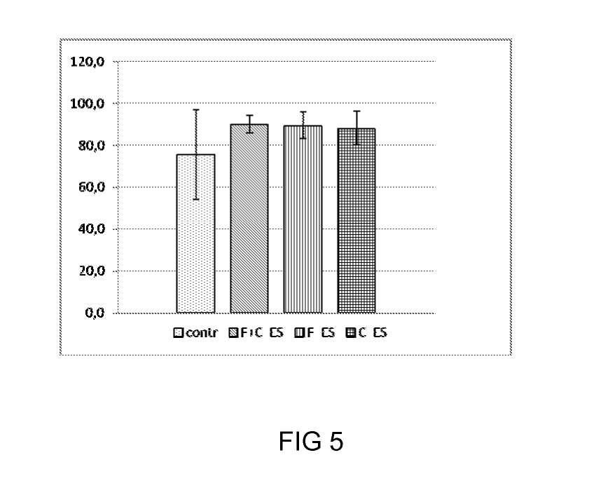

Split-thickness experimental skin wounds where fibrinogen-treated and untreated wounds were compared in 10 days follow-up period. As a result, wound healing was more effective (p=0.03) using fibrinogen-chitosan scaffold than in control wounds (repair at 90.0±4.19% versus 75.5±21.40% of skin surface). The healing of the wounds is visually presented in FIG. 4. Comparative histological evaluation of skin wounds treated with fibrinogen-chitosan scaffold had higher amount of fibroblast-like cells and more signs of epithelization compared to control wounds. Data obtained in Example 2 are provided in Table 1 and schematically presented in FIG. 5.

TABLE 1
Wound healing percentage at the end of experiment
(day 10). WH % = ((A₀ − A_{scab})/A₀) × 100; A₀—original wound area,
A_{scar}—scab area.

| | | WH % | | |
|---|---|---|---|---|
| Rat | | Fibrinogen + | WH % | |
| no. | WH % Control | Chitosan | Fibrinogen | WH % Chitosan |
| 11 | 85.7 | 89.0 | 94.8 | 92.5 |
| 12 | 96.5 | 93.2 | 96.5 | 90.1 |
| 13 | 55.3 | 87.1 | 83.9 | 87.8 |
| 14 | 41.9 | 90.6 | 81.2 | 75.7 |
| 15 | 46.7 | 95.6 | 89.7 | 83.9 |
| 21 | 91.9 | 96.5 | 98.2 | 97.4 |
| 22 | 65.6 | 84.7 | 95.0 | 95.4 |
| 23 | 93.9 | 91.4 | 88.4 | 77.0 |
| 24 | 97.6 | 87.3 | 84.6 | 97.9 |
| 25 | 79.5 | 84.8 | 82.2 | 82.7 |
| Average | 75.5 | 90.0 | 89.5 | 88.0 |
| SD | 21.40 | 4.19 | 6.34 | 8.04 |

### Example 3

### Electrospun Chitosan-Fibrinogen Scaffold for Cell Cultivation

The scaffold disclosed in the present invention was used as a substrate usable in in vitro cultures to cultivate cells for different purposes.

Biocompatibility of the chitosan-fibrinogen scaffold (CFS) was evaluated in vitro by measuring the metabolic activity of fibroblasts cultured on the scaffold for 2-14 days.

The electrospun nanofibers of pure chitosan and fibrinogen-chitosan composition (cut into disks of 1.9 mm² to fit into the well of a 24-well tissue culture plate (cat. 3526, lot. 11500002, Corning Incorporated, Corning, N.Y., USA)) were neutralized and sterilized before cell seeding. Neutralization was carried out in saturated sodium carbonate (Na₂CO₃) solution for 3 hours at room temperature (RT). After that scaffolds were rinsed in distilled water until neutral pH was reached. For sterilization, nanofibres were soaked in 70% ethanol for 2 hours and thereafter rinsed 3 times in phosphate buffered saline (PBS at RT). Prior to cell seeding, scaffolds were kept in culture medium DMEM (Cat. E15-843, Lot. E84310-0274, PAA Laboratories, Austria) with 10% FBS (FAA Laboratories, Austria) for 1 hour at RT. Human dermal fibroblast at their fourth passage obtained from Institute of Cellular and Molecular Biology of the University of Tartu (HF 08/01) were seeded on the scaffolds placed on the bottom of tissue culture plate (TCP) wells. Cells were seeded on 9 electrospun chitosan scaffolds and 9 electrospun fibrinogen-chitosan (50:50) scaffolds. Seeding density was 10 000 cells per well in culture medium (DMEM). One hour after seeding each culture well was gently topped up with 0.4 ml culture medium. This was done to enable cell attachment to scaffolds and prevent wash off of cells from scaffolds. The cultures were maintained in incubator at 37° C. with 5% CO₂. Every 2 days the culture medium (0.4 ml) was changed to facilitate optimal growth conditions. On days 2, 4, 8 and 14 two scaffolds of both materials were harvested for proliferation measurement as described further.

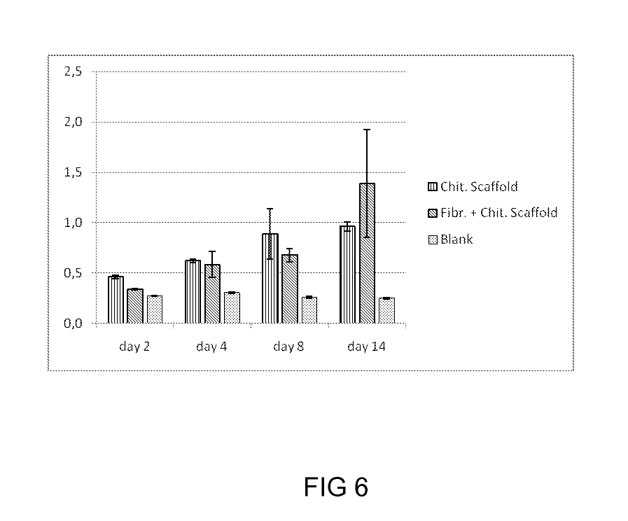

Cellular proliferation was monitored by forming of formazan product (MTS assay)(CellTiter 96 Aqueous One Solution Cell Proliferation Assay, Promega Corporation, WI, USA). The 3-(4,5-dimethylthiazol-2-yl)-5-(3-carboxymethoxyphenyl)-2-(4-sulfophenyl)-2H-tetrazolium (MTS) is bioreduced by cells into a colored formazan product that is soluble in tissue culture medium and can be observed at 490 nm. The culture medium was removed and the cultures were washed with PBS. 400 μl serum free DMEM medium and 80 μl MTS solution were added to each sample followed by incubation at 37° C. for 1.5 hours. The obtained coloured solution was put into 96-well plates and the samples were analyzed using microplate reader at 490 nm. The data about cell proliferation are presented in FIG. 6.

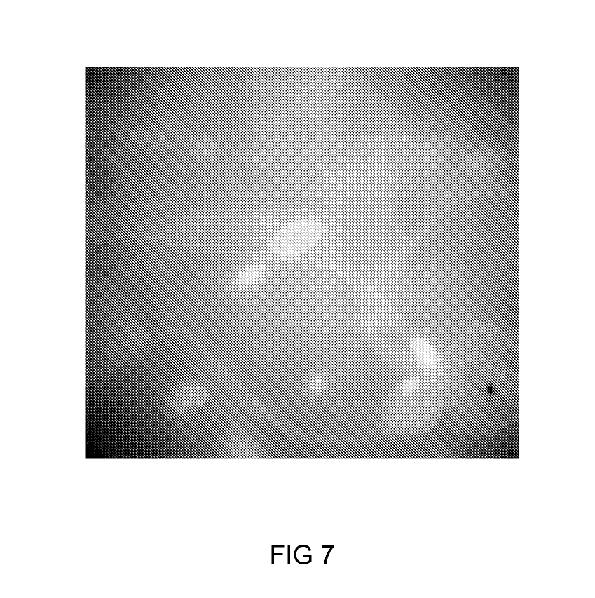

In this experiment it was confirmed that the electrospun fibrinogen-chitosan nanofibers support cell attachment and proliferation indicating their good biological properties. Also it can be seen that organic solvents (hexafluoropropanol and trifluoroacetic acid) used for manufacturing of the scaffold had no toxic effects on the cells attached to the scaffold in vitro during the whole cell cultivation experiment. The results of the experiment are shown in FIG. 7.

Thus, the scaffold obtained by simultaneous electrospinning of the mixture of the solutions of fibrinogen and chitosan is useful for providing a framework for cell attachment and can be applied in cell culture, organ culture, as well as for in vitro and/or in vivo modelling of tissues and organs.

1. A method for the preparation of a durable haemostatic scaffold with enhanced bioadhesivity comprising simultaneously electrospinning fibrinogen and chitosan.

2. The method of claim 1, wherein said method forms fibres of fibrinogen and chitosan, and wherein said fibres are exposed to the inner and outer surface of said scaffold and define gaps of size sufficient for eukaryotic cells to attach and proliferate.

3. The method of claim 1 wherein said scaffold is produced by simultaneous electrospinning of a fibrinogen solution and a chitosan solution in the absence of biocatalysts biocatalysts that promote the polymerization reaction of fibrinogen into fibrin.

4. The method of claim 3, wherein the solvents of fibrinogen and chitosan are halogenated alcohol(s) and halogenated acid(s).

5. The method of claim 4, wherein the solvent of fibrinogen is a 90:10 mixture of hexafluoropropanol/trifluoroacetic acid and the solvent of chitosan is trifluoroacetic acid.

6. The method of claim 1, wherein electrospinning is performed at a voltage ranging from 5 to 30 kilovolts (kV).

7. (canceled)

8. (canceled)

9. (canceled)

10. The method of claim 1, wherein fibrinogen is obtained from a non-mammalian vertebrate.

11. The method of claim 10, wherein said non-mammalian vertebrate is fish.

12. The method of claim 11, wherein said fish is salmon.

13. The method of claim 6, wherein said electrospinning is performed at a voltage of 10 kilovolts (kV).

14. A method for the preparation of a durable haemostatic scaffold with enhanced bioadhesivity comprising:
a. preparing a solution of fibrinogen;
b. preparing a solution of chitosan;
c. preparing a mixture of the solutions of steps a. and b.; and
d. electrospinning the mixture of step. c to form said scaffold;
wherein said electrospinning of said mixture is performed in the absence of biocatalysts that promote the polymerization of said fibrinogen.

15. The method of claim 14, wherein step d. is performed at a voltage ranging from 5 to 30 kilovolts (kV).

16. The method of claim 14, wherein step d. is performed at a voltage of 10 kilovolts (kV).

17. The method of claim 14, wherein the fibrinogen solution comprises a solvent of a 90:10 mixture of hexafluoropropanol/trifluoroacetic acid, and the chitosan solution comprises a solvent of trifluoroacetic acid.

18. A haemostatic scaffold prepared by the method of claim 14.

19. The haemostatic scaffold of claim 16, wherein said scaffold comprises fibres of electrospun fibrinogen and chitosan, whereas said fibres are exposed to the inner and outer surface of said scaffold, and said scaffold defines gaps between said fibers of a size sufficient for eukaryotic cells to attach and proliferate.
